(19) United States
(12) Patent Application Publication
(10) Pub. No.: US 2018/0303469 A1
(43) Pub. Date: Oct. 25, 2018

(54) MEDICAL DEVICE FOR CLOSING FISTULAS, INSTRUMENT FOR THE POSITIONING THEREOF, AND METHOD FOR THE POSITIONING AND EXTRACTION OF THE DEVICE USING SAID INSTRUMENT

(71) Applicants: Jose Pablo DIAZ JIMENEZ, Sant Pere De Ribes, Barcelona (ES); Tarek DAMMAD, Santa Fe, NM (US)

(72) Inventors: Jose Pablo DIAZ JIMENEZ, Sant Pere De Ribes, Barcelona (ES); Tarek DAMMAD, Santa Fe, NM (US)

(21) Appl. No.: 15/743,362

(22) PCT Filed: Jul. 10, 2015
(86) PCT No.: PCT/ES2015/070539

§ 371 Date: Jul. 13, 2018

(51) Int. Cl. A61B 17/00; A61B 17/12

(52) U.S. Cl. CPC A61B 17/0057; A61B 17/12104; A61B 2017/00632; A61B 2017/00623; A61F 2210/0019; A61B 2017/00641

(57) ABSTRACT

The medical device has an element including two discs, small (5) and large (6), joined by a central part (7), the structure of the element being determined by a nitinol core (8) with sufficient flexibility to be folded over itself and inserted in a flexible instrument (9) suitable for incorporation in an endoscope or similar device. Also included is a layer (10) of soft material, such as polyurethane, silicone or another polymer that covers the core (8). The core (8) is filamentous and forms at least the two discs (5, 6) via spokes (81) and the central part (7) via a rod (82). The instrument (9) includes a capsule (11) in which the device (1) that is folded over itself is accommodated, and a catheter (12) that can be connected to the capsule (11), with a pushing mechanism (14) for expelling the device (1) from the capsule (11) in a controlled manner

### OBJECT OF THE INVENTION

The invention as stated in the title of this specification, refers to a medical device for closing fistulas, to the instrument for the positioning thereof and the process for positioning and extracting the device with the said instrument, having advantages and characteristics, that will be disclosed in detail thereafter, which mean a significant improvement of the current state of the art.

The object of this invention is concretely focussed in an improved occlusion device to proceed to closing fistulas in the human body of the type consisting in a double button with an intermediate gap having a width similar to the thickness of the wall where the fistula opening is located, made of flexible material that allows its introduction through it, recovering its initial shape and remaining flush against the external wall and the internal surface, if fit, of the trachea, perfectly closing the opening, which is distinguished from other known devices by the innovating material and structure of which it is made that, advantageously, allow a much simpler process of positioning and extraction using a simple endoscopic technique, because it folds over itself and is housed in a cylindric delivering capsule that is connected to a special delivering endoscope that can be inserted through the working channel of an endoscope, this instrument and the positioning and extraction process also being the object of this invention.

### FIELD OF APPLICATION OF THE INVENTION

The field of application of this invention is within the sector of industry engaged in manufacturing medical devices, specially including the positioning instruments and processes and more concretely, those designed for closing fistulas. The invention is generally applicable to any abnormal communication that can be present in the human body such as tracheoesophageal fistula and a urogenital fistula.

### BACKGROUND OF THE INVENTION

As reference to the current state of the art, in addition to other documents that disclose devices of the type involved herein, the patent ES 2 294 930 A1 of one of the applicants is known, which discloses a device to proceed to closing fistulas in the human trachea and adjacent canals that communicate the bronchial tree with the nearby organs, such as the oesophagus, the mediastinum, the pleura or the thoracic wall, that consists in an element of an harmless, homologated and flexible material with a structure in the shape of a double button, one being fully circular and the other with an elliptical shape, both joined by a cylindrical central part, delimiting a gap equivalent, at least, to the thickness of the tracheal wall such button, after it is introduced, thanks to the elliptical shape of the button that is introduced in first place by the fistula opening, closes the fistula thanks to its elasticity, as well on the external face of the cartilaginous surface of the trachea as by the internal face of mucous surface of the mentioned trachea, without any suture is required. In addition, the elliptical button incorporates a dark radiopaque mark and, therefore, traceable by X-rays, with the purpose of allowing tracing and positioning the device.

Then, the said device, despite it basically meets the objectives to which it is designed, shows some aspects that can be improved, mainly derived from the limitation in the flexibility of the buttons, because it is fully made of silicone, which obliges to insert the device by means of rigid bronchoscopy techniques, that means, using rigid instruments that provokes discomfort to the patient and requires sufficient skill from the physician.

Therefore, it would be convenient to avoid the said drawback by improving the structure and flexibility of the device so that it allows its insertion by means of a flexible bronchoscopy technique that, as it is known, allows the access to the airways without intubation or general anaesthesia is required by using a fibrobronchoscope, the objective of this invention being to develop the said improved device that, on the other hand, is applicable to any other type of fistula.

Concretely, the objective of the invention is a device that can be implemented by means of flexible endoscopy and direct vision, by different endoscopists, pneumologists, gastroenterologists and urologists.

On the other hand, it is to be pointed out that, at least the applicants are not aware of the existence of any other medical device for closing fistulas or similar invention that shows the technical and structural characteristics shown by the one claimed herein.

### DESCRIPTION OF THE INVENTION

The medical device for closing fistulas, instrument for the positioning thereof and method for the positioning and extraction of the device using said instrument proposed by the invention, is therefore configured, as a significant novelty within its field of application, because when implementing it, the objectives above mentioned as suitable are satisfactorily achieved, the characterizing details making it possible and distinguishing it being conveniently included in the end claims attached to this specification.

Concretely, what the invention proposes is an improved device for closing fistulas, namely in the human trachea and adjacent canals that consists in an element of harmless, homologated and flexible material the structure of which has two circular or oval discs, one larger than the other, joined to each other by a central part that delimits a gap between them equivalent, at least, to the thickness of the wall with the fistula opening to be closed, which is distinguished by the fact that the said structure comprises a Nitinol core forming the two discs, preferably composed of filaments that determine several spokes equidistant similar to a wheel, preferably three in angle of 120° because this facilitates its folding, as it will be disclosed thereafter and a straight rod joining both discs by their centre, constituting the central part that keeps them separated by the distance required.

With this and thanks to the properties of the said material, that as it is known, is a very flexible alloy of nickel and titanium and that has shape memory, as well the positioning as the extraction of the device, as it will be seen thereafter, result much simpler, in addition that, as it is a metallic material, it directly results radiopaque, avoiding the need to include additional elements to the device to achieve the said opacity.

Further on with the characteristics of novelty of the closing device of the invention, it shall be mentioned that, preferably, at least the discs are fully covered by a layer of soft material, such as polyurethane, that determines their circular or oval shape, although they also could be of silicone or another polymer.

On its hand, the process of positioning the disclosed device, that as it was stated thanks to its structure of flexible material, preferably formed by spokes, results simpler as it can be carried out by means of the technique of simple endoscopy, comprising the use of a specific instrument and by means of the steps to be stated thereafter:

The device, thanks to its flexibility, folds over itself with the spokes of each disc oriented towards opposite ends and the respective vertices facing each other, after what it is introduced in a delivering capsule, bearing in mind that the small disc, designed to remain inserted behind the wall where the fistula is located, remains at the more external point thereof, while the large disc, designed to remain inserted in the front part of the said wall, is placed at the more proximal point of the said capsule.

The said delivering capsule, is a transparent cylindric capsule, which is made of transparent polyurethane or silicone and that is connected to a special delivering catheter that, in turn, can be inserted through the working channel of the endoscope. As the capsule is transparent, the operation of positioning can be perfectly seen through the camera of the said endoscope.

In addition, the delivering capsule, at its proximal end, has retention means that allow to join the distal end of the catheter to it, the said means consisting, preferably, in protrusions having a special shape in which is fit, simply by pressure, the distal end of the catheter itself that has a complementary configuration.

As for the catheter, it is of the type of standard delivery with a diameter that is much smaller than that of the delivering capsule that contains the closing device of the invention and it is distinguished in that the distal end has a special shape that can be connected to the proximal end of the delivering capsule, as it has been disclosed before.

The catheter, in addition, internally incorporates a pushing mechanism, preferably consisting in a piston-like means that, when coupled to the delivering capsule, is capable of pushing the closing device to correctly deposit it in the fistula opening, moving it, at least, to a distance equal to half the diameter of the disc plus the length of the rod forming the connecting central part.

It shall be pointed out that, to facilitate such positioning, this rod that forms the connecting central part between both discs of the device is extended in a length that emerges from the large disc designed to be positioned in the front part of the wall where the fistula opening to be closed is located and that, therefore, it is oriented towards the proximal part of the capsule remaining facing the distal end of the catheter.

The catheter, as it has been said, uses a piston-like means as pushing element which is actuated by means of a button located at the proximal end of the catheter that, when rotating it, moves a control stem forwards in the catheter to be coupled with the rod of the device and to push it forwards out the delivering capsule.

With all that, the steps of the positioning process are as follows:
The supplying catheter is inserted through the working channel of the endoscope until its distal end is out of it.
A delivering capsule having the correct size is selected, the device folded over itself being introduced in it, as it was disclosed, and its proximal end is connected to the distal end of the delivering catheter.
The endoscope with the supplying catheter and the delivering capsule that contains the closing device is inserted in the hole of the human body (for example, through the vocal chords to the trachea) until the location of the fistula is identified.
The distal end of the delivering capsule is positioned just at the beginning of the fistula.
The pushing element of the delivering catheter is actuated pushing as well the first disc of the closing device as the connecting rod within the fistula. This process unfolds the first disc within the canal, remaining flush against the internal wall thereof covering the fistula by the said internal part.
At this point, the second disc of the closing device is about to be also unfolded, under the direct vision of the endoscope. To implement the second disc just on the external wall of the fistula, the set of endoscope, administering catheter and capsule are pulled backwards allowing that the said second disc is unfolded flush against the external wall of the fistula

The final result is that the closing device is located on both sides of the fistula, closing it to avoid the communication of both organs.

As for the process of extraction of the device, it contemplates the following steps:
Using a suitable gripping element, such as a biopsy forceps, that is introduced through the endoscope and allows to pull the closing device fastening it by one of the discs, normally the one located outside, until the other disc is completely folded over itself and crosses the wall of the fistula, with which a small hole remains.
Thereafter, the full device can be extracted outside the body.
Then, the full device can be extracted out of the body.

Logically, as well the device as the instruments used must have variable diameters, depending on the requirements of each case.

The medical device disclosed for closing fistulas, the instrument for its positioning and the process for positioning and extracting the device with the said instrument consist therefore in innovating structures having characteristics unknown up to now for the purpose to which they are designed, reasons that, jointly with their practical usefulness, provide it with sufficient foundation to obtain the privilege of exclusivity applied for.

### DESCRIPTION OF THE DRAWINGS

To complement the description that is been done and in order to assist to a best understanding of the characteristics of the invention, attached to this specification, as an integral part thereof, is a set of drawings in which with illustration and non-limiting purpose the following has been represented:

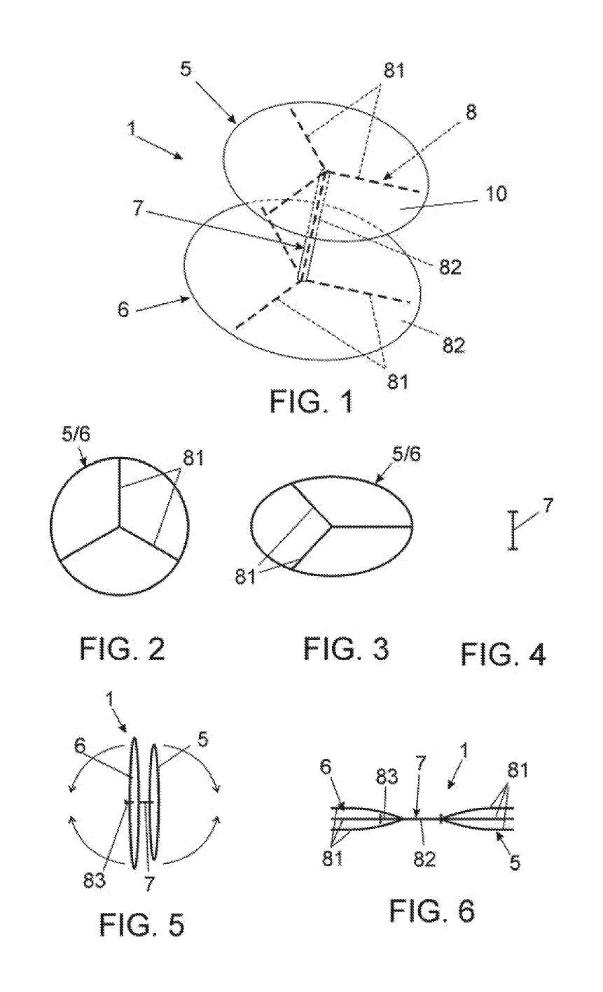

The FIG. 1.—It shows a view in perspective of an example of preferred embodiment of the medical device for closing fistulas, object of the invention, where the parts and elements it comprises can be seen;

The FIGS. 2 and 3.—They show schematic plan views of two examples, circular and oval, respectively, of a preferred configuration of the discs of the closing device of the invention.

The FIG. 4.—It shows a very schematic and elevation view of the rod forming the connecting central part between both discs of the device, according to the invention; and

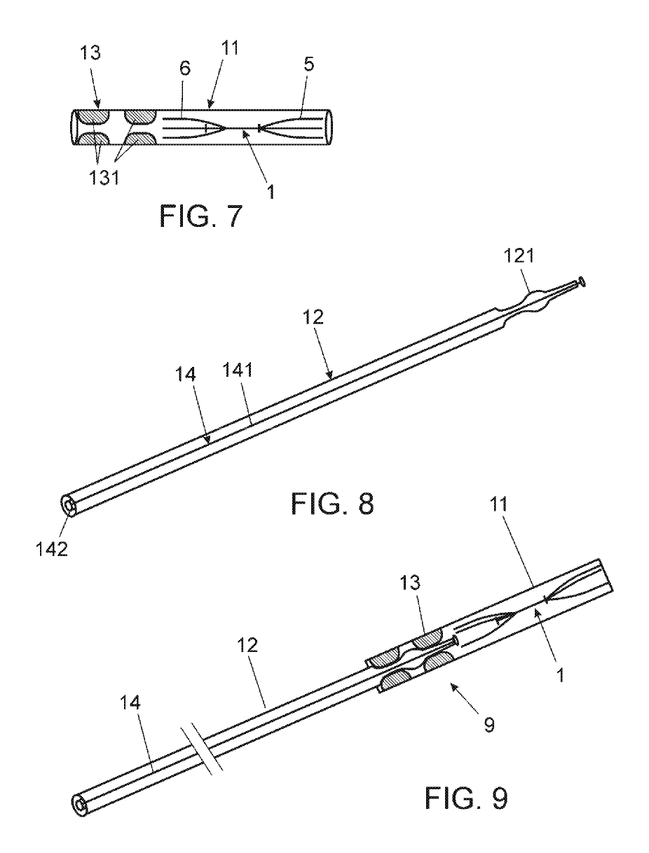

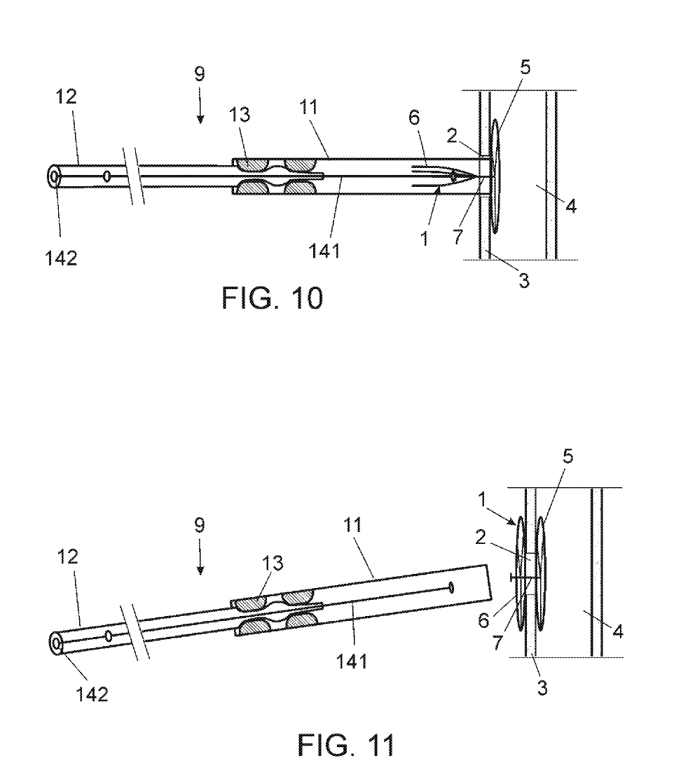

The FIGS. 5 to 11.—They show a series of schematic views of the sequence of the process of positioning the device of the invention and the instrument used in the said process. Concretely, the FIG. 5 shows the folding movement of the discs, the FIG. 6 shows the device after it is folded over itself and before its implementation, the FIG. 7 shows a cross view of the delivering capsule with the device housed within it; the FIG. 8 shows a perspective of the catheter where the internal pushing means can be seen, the FIG. 9 shows the catheter already coupled to the delivering capsule, the FIG. 10 shows the unfolding of the first disc on the internal wall of the fistula after positioning the instrument in the body of the patient with an endoscope and the FIG. 11 shows the extraction of the instrument after the unfolding of the second disc of the closing device on the external wall of the fistula.

### PREFERRED EMBODIMENT OF THE INVENTION

Seen the said figures and according to the numbering adopted, a non-limiting example of the medical device for closing fistulas of the invention and of the instrument used for the positioning thereof can be seen that comprises the parts and elements mentioned and disclosed in details below.

Thus, in the said figures, the device (1) involved can be seen, designed to closing a fistula (2) on the wall (3) of a canal (4) of a human organ, that consists in a manner already known, of an element the structure of which has two circular discs (5, 6), one small (5) designed to remain positioned covering the fistula (2) by the interior part of the wall (3) and one large (6) designed to be positioned by the external part of the said wall (3), both discs (5,6) being joined by a central part (7) that delimits the gap between them at the distance required, with the peculiarity that the said structure has a Nitinol core (8) providing it with a flexibility such that allows its folding over itself and its insertion in a specific flexible instrument (9) (see FIGS. 9, 10 and 11) that, in turn, is adapted to be incorporated in an endoscope or similar device.

More concretely, the Nitinol core (8) forms, at least, the two discs (5, 6) via filaments, concretely via several spokes (81), preferably three equidistant spokes, joined at their centre and, preferably, it also forms the central part (7) that joins both discs at the said centre, via a straight rod (82).

In addition, preferably, the said rod (82) has an extension (83) that emerges by the external part of one of the discs, preferably that of the large disc (6) serving as support base to facilitate its positioning by means of the instrument (9).

Anyway, in the preferred embodiment, at least the two discs (5,6) of the device (1) formed by the Nitinol core (8) are in addition fully covered by a layer (10) of soft material, of polyurethane, silicone or other polymer, circular or oval shaped, although, in the preferred option of the invention, also the central part (7) formed by the straight rod (82) of the Nitinol core (8) is covered by the said layer (10) of soft material.

Referring to the FIGS. 2 to 11, the main steps of the process of positioning the device (1) of the invention using the said flexible instrument (9) is seen, which essentially comprises a delivering capsule (11), suitable to house the closing device (1), folded over itself, and a catheter (12) that is connected to the capsule (11) via retention means (13), provided in turn with a pushing means (14) to push and expel under control the device (1) and that this way, can be inserted through the working channel of an endoscope or other similar instrument.

The capsule (11) consists of a hollow cylinder open by both ends and transparent, preferably of transparent polyurethane or silicone and that is connected by its proximal part to the distal end of the catheter (12) by means of the said retention means (13) that, preferably, comprise protrusions (131) between which there exists a gap where a complementary thickening (121), provided at the said distal end of the catheter (12) is fitted under pressure.

As for the catheter (12), it has a diameter much smaller than the one of the capsule (11) and the pushing means (14), preferably, consists of a piston-like element formed by the stem (141) that, when screwing a button (142) of the proximal end of the catheter (12) moves towards the distal end of the capsule (11) being capable to push the closing device (1) housed within it, preferably affecting the extension (83) of the rod (82).

As for the steps of the process for positioning the closing device (1) they are essentially as follows:
Inserting the catheter (12) through the working channel of the endoscope or similar instrument until its distal end is out of it.
Choosing a delivering capsule (11) having a correct size, introducing the device (1) folded over itself in it and connecting its proximal end to the distal end of the catheter (12).
Introducing the device (1) folded over itself in the capsule (11) with the spokes of each disc oriented towards opposite ends and with their respective vertices facing each other, having care that the small disc (5) remains at the more external point thereof, while the large disc (6) remains at the more proximal point of the capsule (11).
Inserting in the relevant hole of the human body the endoscope or similar with the catheter (12) and the capsule (11) that contains the device (1) until positioning the distal end of the capsule (11) just at the beginning of the fistula (2).
Actuating the pushing means (14) of the catheter (12) by pushing the first disc (5) of the device (1) within the fistula, that is unfolded remaining flush against the internal wall of the canal (2).
Pulling backwards the set of the endoscope or similar instrument, catheter (12) and capsule (11), allowing that the second disc (6) is unfolded flush against the external wall of the fistula.

As for the process of extraction of the device (1) it consists of:
Using an incorporated suitable gripping means, such as a biopsy forceps, that is introduced through the endoscope and pulling the device (1) fastening it with one of the discs until the other disc (5) is fully folded and crosses the wall of the fistula.
Extracting the full closing device (1) out of the body.

The nature of this invention being sufficiently disclosed as well as the way of implementing it, it is not deemed necessary to extend any longer this explanation in order that any person skilled in the art comprises the extent and the advantages derived from it, and it can be reimplemented in other embodiments differing in detail of the indicated for example purpose provided that the fundamental is not altered, changed or modified.

1. A medical device for closing fistulas, namely designed for closing a fistula (2) in the wall (3) of a canal (4) of a human organ that, consisting in an element the structure of which shows two circular or oval discs (5,6), one small (5) designed to remain positioned covering the fistula (2) by the internal part of the wall (3) and one large (6) designed to be positioned by the external part of the said wall (3) the said discs (5, 6) being joined by a central part (7) that delimits the gap between both to the distance required, is characterized in that the said structure comprises a Nitinol core (8) having sufficient flexibility to be folded over itself and inserted in a flexible instrument (9) suitable for the incorporation in an endoscope or similar device and a layer (10) of soft material such as polyurethane, silicone or other polymer that covers the central part (8).

2. The medical device for closing fistulas, according to claim 1 characterized in that the Nitinol core (8) is filamentous.

3. The medical device for closing fistulas, according to the claim 2 characterized in that the Nitinol core (8) forms, at least, the two discs (5, 6) via several spokes (81) joined by their central part.

4. The medical device for closing fistulas, according to claim 3 characterized in that the Nitinol core (8) forms the two discs (5, 6) via three spokes (81) equidistant at 120°.

5. The medical device for closing fistulas, according to claim 2 characterized in that the Nitinol core (8) forms the central part (7) that joins both discs via a rod (82).

6. The medical device for closing fistulas, according to claim 5, characterized in that the central part (7) is a rod (82) having an extension (83) emerging by the external part of one of the discs (6) serving as supporting basis to facilitate its positioning with the instrument (9).

7. The medical device for closing fistulas, according to claim 1 characterized in that, at least, the discs (5, 6) formed by the Nitinol core (8) are fully covered by a layer of soft material (10).

8. The medical device for closing fistulas, according to claim 7 characterized in that the central part (7) of the Nitinol core (8) is also covered by a layer of soft material (10).

9. The medical device for closing fistulas, according to claim 7 characterized in that the layer of soft material (10) is preferably of polyurethane.

10. An instrument for positioning the device (1) for closing fistulas characterized in that the said instrument (9) is flexible and suitable for the incorporation in an endoscope or similar device and comprises a delivering capsule (11) suitable for housing the closing device (1) folded over itself and a catheter (12) that is connected to the capsule (11), which has a pushing means (14) to push and expel under control the device (1) out of the capsule (11) and that, in turn, can be inserted through the working channel of the endoscope or similar instrument.

11. The instrument, according to claim 10, characterized in that the catheter (12) is connected to the capsule (11) though retention means (13).

12. The instrument according to the claim 10, characterized in that the capsule (11) is a hollow cylinder open at both ends.

13. The instrument according to claim 10, characterized in that the capsule (11) is transparent.

14. The instrument, according to claim 10, characterized in that the capsule (11) is preferably of transparent polyurethane or silicone.

15. The instrument, according to claim 10, characterized in that the said retention means (13) that connect the capsule (11) by its part proximal to the distal end of the catheter (12), comprise protrusions (131) between which there exists a gap where a complementary thickening (121) provided at the said distal end of the catheter (12) is fitted under pressure.

16. The instrument, according to claim 10, characterized in that the catheter (12) has a diameter much smaller than the capsule (11).

17. The instrument, according to claim 10, characterized in that the pushing means (14) of the catheter (12) consists in a piston-like means formed by a stem (141) that, when screwing a button (142) of the proximal end of the catheter (12) moves towards the distal end of the capsule (11) being capable to push the closing device (1) housed within it.

18. A process for positioning a device (1) for closing a fistula characterized in that it comprises the following steps:
inserting a catheter (12) through a working channel of the endoscope or similar instrument until a distal end of the catheter (12) is outside the endoscope;
selecting a delivering capsule (11) having a correct size, introducing a device (1) folded over itself into it and connecting a proximal end of the device to the distal end of the catheter (12);
inserting in a relevant hole of a human body the endoscope or similar with the catheter (12) and a capsule (11) containing the device (1) until positioning the distal end of the capsule (11) just at the beginning of the fistula (2);
actuating a pushing means (14) of the catheter (12) by pushing a first disc (5) of the device (1) within the fistula (2), that is unfolded remaining against an internal wall of the the fistula; and
pulling backwards from an assembly of the endoscope or similar instrument the catheter (12) and capsule (11), allowing that a second disc (6) is unfolded flush against the external wall of the fistula (2)

19. The process for positioning, according to claim 18, characterized in that the closing device (1) is introduced folded over itself in the capsule (11) with the spokes (8) of each disc oriented towards opposite ends and with their respective vertices facing each other, having care that the small disc (5) remains at the more external point thereof, while the large disc (6) remains at the more proximal point of the capsule (11).

20. A process for extracting a medical device (1) for closing fistulas in a body, characterized in that it comprises:
using an incorporated suitable gripping means that is introduced through an endoscope and pulling the medical device (1) fastening the medical device (1) with one of the discs until the other disc (5) is fully folded and crosses the wall of the fistula; and
extracting the medical device (1) out of the body.
